(19) United States
(12) Patent Application Publication
(10) Pub. No.: US 2015/0051807 A1
(43) Pub. Date: Feb. 19, 2015

(54) Method For Operating A Vehicle, A Vehicle And A Detection Assembly

(71) Applicants: Hiren Harshadrai Desai, Orchard Lake, MI (US); Karan Hiren Desai, Orchard Lake, MI (US)

(72) Inventors: Hiren Harshadrai Desai, Orchard Lake, MI (US); Karan Hiren Desai, Orchard Lake, MI (US)

(21) Appl. No.: 13/987,601

(22) Filed: Aug. 13, 2013

(51) Int. Cl. B60K 28/06; G01N 33/98; G01N 33/497

(52) U.S. Cl. CPC B60K 28/063; G01N 33/4972; G01N 33/98; B60K 2028/003
USPC 701/99

(57) ABSTRACT

A vehicle 10 including sensors 40-48 and 50 and a processor assembly 30 which, when operational according to one or more strategies, reduce the likelihood of the vehicle 10 being operated by an inebriated driver and the strategy may be portable and suitable for aftermarket applications.

### GENERAL BACKGROUND

1. Field of the Invention

The present invention generally relates to a method for operating a vehicle, a vehicle and to a detection assembly, and more particularly to strategies and implementations for reducing the likelihood of a vehicle from being started by an inebriated person.

2. Background of the Invention

Vehicles which are operated by inebriated individuals (those having a blood alcohol level above a certain legal threshold) have been known to be at a relatively high risk to be involved in an accident. While legal penalties for operating a vehicle while being inebriated are typically severe, there are often imposed only after a tragic accident occurs.

There is a need for new and improved strategies and techniques to reduce the likelihood of allowing an inebriated individual to start and operate a vehicle and the present invention provides these.

### SUMMARY OF THE INVENTION

It is a first non-limiting object of the invention to provide strategies and techniques which overcome the previously delineated drawbacks of current strategies and techniques.

It is a second non-limiting object to provide strategies and techniques to reduce the likelihood of a vehicle being started by a inebriated or “drunk driver”.

According to a first non-limiting aspect of the present invention, a method for operating a vehicle is provided and includes the steps of providing a plurality of first sensors; placing said plurality of first sensors upon a portion of said vehicle; providing a second sensor; analyzing a first breath by use of said plurality of said first sensors; analyzing a second breath by use of said second sensor only if said plurality of said first sensors collectively determine that said first breath evidences an alcohol content above a certain threshold; and preventing said vehicle from being started if said second sensor determines that said second breath evidences an alcohol content above said certain threshold.

According to a second non-limiting aspect of the present invention, a vehicle is provided and includes a steering wheel; an engine; a starter; at least one sensor connected to said steering wheel; a second sensor; a processor which is coupled to said starter, to said at least one sensor, and to said second sensor, wherein said at least one sensor and said second sensor analyze breath and based upon said analysis cooperatively cause said processor to selectively prevent said starter from starting said engine.

According to a third non-limiting aspect of the present inventions, a detection assembly is provided and includes a pliable sleeve; a plurality of sensors operatively placed upon said sleeve; a second sensor; and a processor assembly which is coupled to said plurality of sensors and to said second sensor and which prevents an engine from being started when said plurality if sensors and said second sensor collectively identify an inebriated individual.

These and other aspects, features, and advantages of the present inventions will become apparent from a reading of the following detailed description of the preferred embodiment of the invention, including the subjoined claims, and by reference to the following drawings.

### BRIEF DESCRIPTION OF THE DRAWINGS

FIG. 1 is a partial side view of a vehicle made in accordance with the teachings of the preferred embodiments of the inventions.

FIG. 2 is a flowchart comprising the sequence of steps associated with the methodology of the preferred embodiment of the inventions.

FIG. 3 is a perspective view of a detection assembly which is made in accordance with the teachings of the preferred embodiment of the inventions.

### DETAILED DESCRIPTION OF THE PREFERRED EMBODIMENT OF THE INVENTIONS

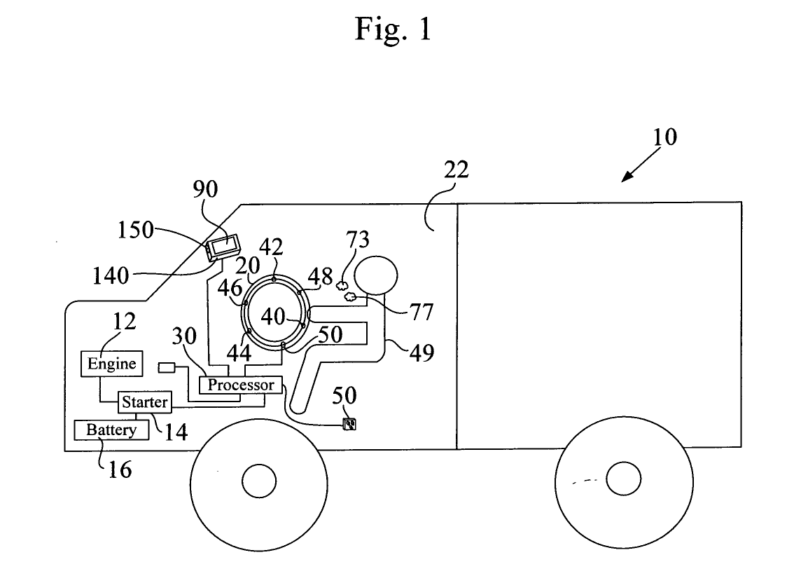

Referring now to FIG. 1, there is shown a vehicle 10 which is made in accordance with the teachings of the preferred embodiment of the inventions.

Particularly, vehicle 10 includes an engine 12 and a starter assembly 14 which is coupled to the engine 12 and which selectively starts the engine 12. The vehicle 10 further includes a battery 16 which is coupled to the starter 14 and which provides a source of operational electrical energy to the starter 14. The vehicle 10 further includes a steering wheel 20 which is operatively disposed within a passenger compartment 22. The vehicle 10 may comprises any type of known and commercially available vehicles available in the marketplace today and only those vehicular portions necessary for the description of the inventions are delineated here.

Vehicle 10, according to the most preferred embodiment of the invention, includes a processor assembly 30 which is operable under stored program control and which may comprise substantially any type of commercially available processor, such as that which is known as an “I5” and which is available from the Intel® corporation. The vehicle 10 further includes a plurality of breath analysis type sensors 40-48 which in one non-limiting embodiment, are disposed upon the surface 50 of the steering wheel 20 and which are in communication with a driver or operator 49 of the vehicle 10. In one embodiment, the sensors 40-48 are substantially identical and each comprise a commercially available model “MQ-3” Semiconductor Sensor which is described at and which may be ordered from the website https://www.dhgate.com/. The sensors 40-48 may be each secured upon the steering wheel 20 by respective fasteners or by an adhesive, or may be integrally molded onto surface 50 of the steering wheel 20 during the formation of the steering wheel 20. The sensors 40-48 are coupled to the processor assembly 30 and the processor assembly 30 is coupled to the battery 16 and receives operational electrical energy from the battery 16. Moreover, the vehicle 10 further includes a second sensor 50 which is coupled to the processor assembly 30 and which comprises a commercially available model “AL900 Alcoscan” breathe sensor which is described at and which may be obtained from https://www.precisionbreathalyzer.com/. It should be appreciated that any number of sensors 40-48 and 50 may be utilized within the vehicle 10 and that sensor 50 is in communication with the operator or driver 49.

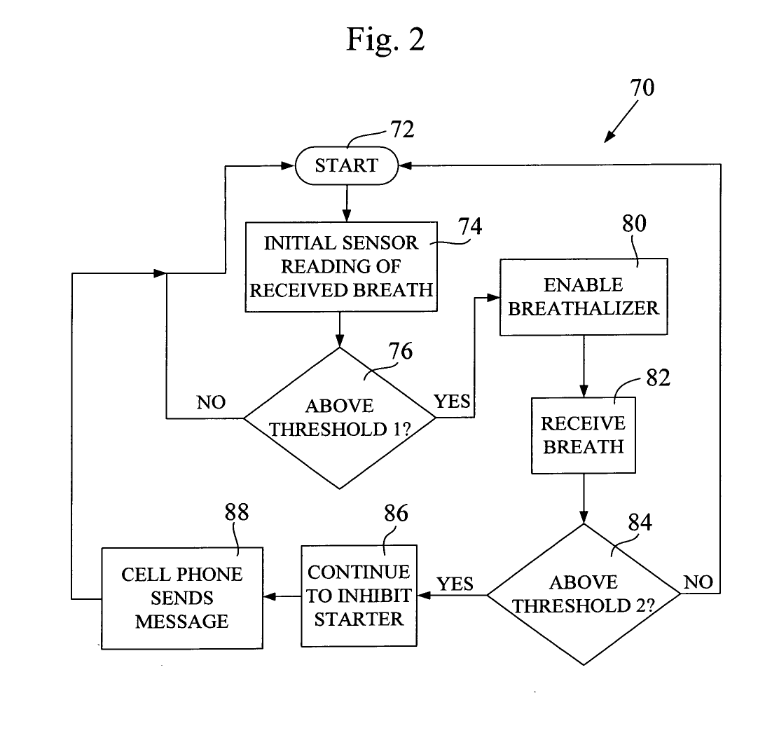

Referring now to FIG. 2, there is shown a flow chart 70 which illustrates the sequence of operational steps involve in the methodology of the inventions.

Particularly, the operation beings with the initial or “start” step 72 which denotes the operational readiness of the sensors 40-48, 50, and the processor assembly 30. Step 72 is followed by step 74 in which sensors 40-48 collectively receive breath 73 from the operator 49. Step 76 follows step 74 and, in this step 76, the sensors 40-48 determine whether the received breath 73 evidences that the operator 49 has a level of alcohol above a certain limit. In one non-limiting embodiment, each of the sensors 40-48 must determine that this limit or threshold has been exceeded in order for step 80 to follow step 76. Alternatively, step 76 is followed by step 72.

In step 80, the processor assembly enables the sensor 50, by allowing electrical energy to be sourced to the sensor 50 from the battery 16, and step 82, which follows step 80, requires that the operator 49 provide a breath 77 to the sensor 50. Step 84 follows step 82 and, in this step 84, the sensor 50 determines whether the received breath evidences that the operator has an alcohol level above the certain pre-defined threshold. If such a determination is made, then step 84 is followed by step 86 in which the processor assembly 30 continues to inhibit the operation of the starter assembly 14, thereby continuing to prevent the engine 12 from starting and thereby preventing the inebriated driver 49 from operating the vehicle 10. Alternatively, step 84 is followed by step 72. That is, normally the processor 30 inhibits the operation of the starter 14 until the sensors 40-48 and 50 allow the starter 14 to become selectively activated. The respective thresholds of steps 76 and 84 may be substantially identical but are not required to be substantially identical.

Step 88 follows step 86 and, in this step 88, the processor assembly 30, which may be in “Bluetooth” type communication with the cellular telephone 90 of the operator 49, causes the cellular telephone to text a warning message to a predefined cellular telephone. The message and targeted number may be stored within the processor assembly 30. Step 88 is followed by step 72.

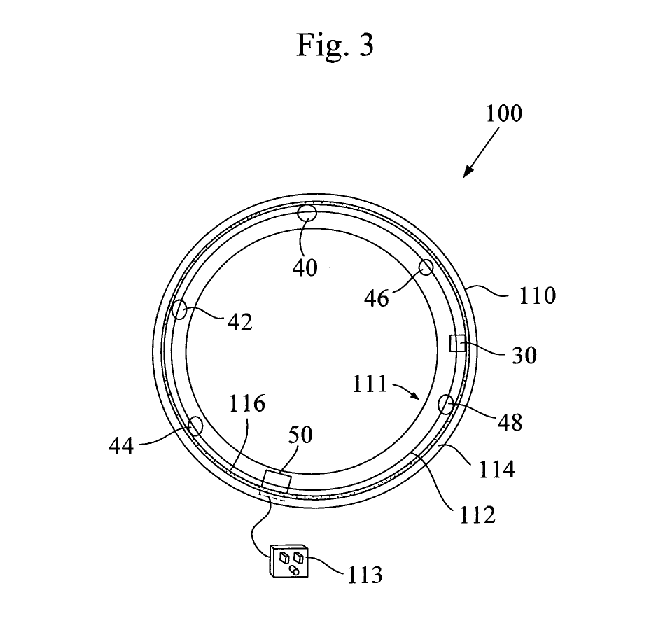

In another non-limiting embodiment of the invention, as is best perhaps shown in FIG. 3, a kit 100 may be provided for use with existing vehicles in an aftermarket strategy for reducing the likelihood of inebriated individuals from operating a vehicle.

The kit 100 includes a pliable sheath or sleeve 110 having sensors 40-48 operatively disposed upon the surface 111. Sensor 50 is also operatively disposed upon the surface 111 and the processor assembly 30 is included within the body 114 of the sheath 110. Bus 112 which resides within the sheath 110 connects the sensors 40-48 and 50 together and is further connected to a portable and rechargeable battery 113. The bus 112 further connects the processor assembly 30 to the sensors 50 and 40-48 and to battery 113. The sheath 110 is adapted to be placed over the steering wheel 20. That is, the open surface 116 of the sheath 110 is placed upon the steering wheel 20 and the steering wheel 20 is thereby resident within the sheath 110.

In yet another non-limiting embodiment of the invention, a cellular telephone dock 140 is placed within the driving compartment and coupled to the processor assembly 30. The cellular telephone 90 of the use 49 must be placed within the dock 140 in order to allow the vehicle 10 to start and if removed a loud audible warning is given. The dock 140 is coupled to processor 30. That is, in one embodiment, the dock 140 includes a sound generator 150 which, when the processor 30 detects a removal of the cellular telephone 90, is activated by having electrical energy communicated to it by the processor 30. In this manner, the likelihood of the driver 49 texting and driving is reduced. The advantage of the initial sensors 40-48 is that you do not have to breathe into the sensor 50 every time you attempt to start your car. The initial sensor 40-48 will do a preliminary test for alcohol, so as to not be a burden on the driver and it will do an initial alcohol level test subconsciously.

It is to be understood that the present inventions are not limited by the exact construction and methodology illustrated in this Application but that various changes and modifications may be made to the inventions as they are delineated in the following claims with departing from the spirit and the scope of the inventions. The foregoing strategy is not limited to use within a vehicle but may be used within and/or as part of an aircraft

What is claimed is:

1) A method for selectively allowing a vehicle having a steering wheel to be operated, said method comprising the steps of providing first sensors; steering wheel; providing a second sensor; analyzing first breath by use of said first sensors; analyzing second breath by use of said second sensor only if said first sensor collectively determines said first breath has a certain first attribute; and preventing said vehicle from being operated only if said second sensor determines said second breath has a certain second attribute.

2) The method of claim 1 wherein said first attribute is substantially similar to said second attribute.

3) A vehicle comprising a steering wheel; an engine; a starter which selectively starts engine; at least one sensor resident within said steering wheel; a second sensor; a processor which is coupled to said starter, to said first sensor, and to said second sensor wherein said first and second sensor analyze breath and based upon said analysis jointly cause said processor to selectively prevent said starter from starting said engine.

4) The vehicle of claim 3 further comprising a cellular telephone dock which detects the presence of a cellular telephone.

5) A detection assembly comprising a pliable sheath; a plurality of sensors operatively placed upon said sheath; a second sensor; and a processor assembly which is coupled to said plurality of sensors and to said second sensor and which prevents an engine from being started when said plurality of sensors and said second sensor collectively identify an inebriated individual.
